(19) United States
(12) Patent Application Publication
(10) Pub. No.: US 2009/0177221 A1
(43) Pub. Date: Jul. 9, 2009

(54) Endovascular implant for occlusion of a blood vessel

(76) Inventor: Bernhard Kramann, Homburg (DE)

Correspondence Address: DONN K. HARMS;PATENT & TRADEMARK LAW CENTER, SUITE 100, 12702 VIA CORTINA, DEL MAR, CA, 92014, US

(21) Appl. No.: 11/988,672

(22) PCT Filed: Jul. 11, 2006
(86) PCT No.: PCT/DE2006/001194

§ 371 Date: Jan. 26, 2009

(30) Foreign Application Priority Data
Jul. 11, 2005 (DE) 10 2005 032 308.1

(51) Int. Cl. A61M 29/00; A61F 2/06

(52) U.S. Cl. 606/195; 623/1.17; 623/1.18

(57) ABSTRACT

An expandable endovascular implant (10) of the stent type is described, which is suitable in particular for occlusion of a blood vessel (31). The implant comprises a circumferential tubular wall extending about a longitudinal axis and composed of a radially expandable, flexible lattice structure, said wall enclosing a cylindrical hollow space with a cross section, characterized in that the cylindrical hollow space defined by the wall is narrowed at least one location along its axis.

The invention relates to an endovascular implant for occlusion of a blood vessel in accordance with the type disclosed in the preamble of claim 1, a method for its production and a medical kit, which contains such an implant.

Such an implant is inserted, for example, by means of a catheter into a blood vessel that exhibits a shunt between an artery and a vein with increased flow, so that the implant makes it possible to position with precision metering the occlusion material without the risk of the material being dislodged. Furthermore, such an implant allows a meterable constriction of the vessel over a definable period of time up to the final occlusion in order to accustom the target organ to exsanguination during this period and to avoid tissue necrosis.

A living human or even animal organism is fed in essence by an extensive system of bifurcating vessels. In this system pathological shunts that are of hereditary origin or are caused by injuries may develop. Arteriovenous stents are perhaps rare diseases, but they pose a potential danger as a function of their size and the target organs. The most prevalent arteriovenous shunts occur in certain cardiac defects and can be treated by either surgical intervention or by inserting occluding membranes by means of vascular catheters. Another large group of arteriovenous shunts constitutes vascular malformations of the pulmonary arteries with shunts to the pulmonary veins. They are frequently observed in the so-called Osler disease. Such shunts can cause the heart to work harder, which in turn leads to premature heart failure. Another complication of concern constitutes heart attacks and brain abscesses, which can be caused by small clots being dragged out of the venous circulation area over the shunts into the arterial circulation area, since the filtering function of the lungs can no longer be effective. Finally, such vascular malformations can also dilate aneurysm-like and burst, so that in these cases the outcome is life-threatening hemorrhaging.

The prior art method for treating such arteriovenous malformations with shunts between the pulmonary artery and the pulmonary vein is to insert embolization material via catheters into a branch of a pulmonary artery, which empties into such a malformation with an arteriovenous shunt. Prior to emptying into the malformation, this artery sends regular branches into the lung tissue, which ought to be protected as much as possible, in order to avoid the clinical symptoms of pulmonary embolism. Thus, the embolization material has to be deposited directly in front of the mouth of the malformation. The malformation exhibits a glomerular, sometimes an aneurysm-like dilation of the vessel, which then continues as the vein following a 180 deg. change in direction in the sense of a shunt and runs directly to the left atrium of the heart. Since it involves a shunt, a high blood flow usually prevails in the afferent branch.

The embolization material, which is used to plug this vascular malformation, comprises platinum-containing spirals—the coils—which are inserted in an elongated state into the catheter, and which form—upon emerging from the tip of the catheter—a type of coil, which in the ideal case blocks the vessel directly in front of the transition of the artery into the aneurysm-like expansion. Such coils for the formation of clots are disclosed, for example, in the WO93/16650. Usually one spiral is insufficient, so that a plurality of spirals is necessary in order to totally obstruct the flow of blood. Not infrequently the afferent branch of the pulmonary artery is greatly expanded. The expansion may range from 20 to 30 mm and more.

However, the aforementioned coils suffer from the drawback that the physician cannot accurately foresee the size to which the coil to be placed in the respective blood vessel will unfold. At this stage he is faced with the difficulty of selecting the dimensions of the spiral in such a manner that it is neither undersized nor oversized. If the spiral is undersized, there is the high risk that it cannot lock in the vessel on account of the strong flow, but rather is dislodged and removed from the malformation into the pulmonary vein and, thus, flows over the left heart into the cardiovascular system. This means that the spiral makes its way first through the left atrium and then through the left ventricle. Thereupon, it enters into the large thoracic aorta and then migrates randomly into a bifurcating artery. If this bifurcating artery is unfortunately a carotid artery, the consequence is a heart attack. There is also great concern that the spiral will be dislodged and float away into an intestinal vessel, since that could lead, for example, to an infarction of the intestines or the kidneys.

If, on the other hand, the spiral is oversized in its dimensions, it cannot unfold, as intended, in the shape of a coil, but rather remains elongated in the too narrow vascular segment. If this is the case, the lateral branches, which lead to the still healthy portions of the lung, are also blocked. For this reason the physician must have a lot of experience so as to avoid complications. If a significantly expanded vessel shows a strong blood flow that increases the risk of the spiral being dislodged, the spiral has to be anchored with its lead section in one of the healthy efferent lateral branches, so that the major portion of the spiral can lie in the pathologically expanded vessel segment without being dislodged, even if the spiral is relatively undersized. Then the subsequent spirals can be deposited directly into the expanded vessel. Thus, the first spiral serves to block the flow and then to bring the following spirals into a stable position. Of course, such a maneuver is not simple, since the pulsation and the breathing motions make it difficult to guide the vascular catheter.

Another way to avoid complications is to use controlled detachable spirals, which are not totally detached from the catheter until it is clear that their larger segment, which has already emerged from the catheter, can be securely wedged in the vessel. Said spirals can be detached from the catheter by means of a thread or also galvanically. These products are in practical use, but they are very expensive and also not totally reliable. Thus, for example, the detachment by a thread can become a problem if the catheter has to perform a number of turns in its passage through the right heart and the pulmonary arteries.

The object of the present invention is to overcome the above-described problems and to provide an endovascular implant for occlusion of a blood vessel. After placement at the desired location, said implant will not dislodge unintentionally and, thus, cause serious subsequent complications. In particular, the implant shall also be suitable for blood vessels with a large diameter. In addition, it shall be guaranteed that in the event that blood-thinning medications—for example, an antithrombotic agent—are administered, the implant cannot dislodge from its location.

This object is solved by the features disclosed in claim 1.

The invention provides an endovascular, stent-like expanding implant, which is suitable, in particular, for occlusion of a blood vessel. The implant comprises a circumferential tubular wall, which extends about a longitudinal axis and is made of a radially expandable, flexible lattice structure. Said wall encloses a cylindrical hollow space with a cross section. The implant is characterized in that the cylindrical hollow space, which is defined by the wall, exhibits a constriction at least at one location along the axis of said hollow space. The constriction can be configured at a distal end of the tubular wall and/or between its ends.

Such an implant can be inserted in the compressed state into the blood vessel by means of a catheter or a balloon catheter and can be unfolded in situ by dilation of the balloon or by thermal conversion. Hence, the diameter of the blood vessel to be treated can be determined in advance, for example, by angiography; and the diameter of the unfolded tubular implant can be adjusted in such a manner that it can be guaranteed that the implant will rest securely against the wall of the blood vessel. This measure offers the advantage of reducing the risk that the implant will detach itself again at a later date. For this reason the inventive implant is also suitable for blood vessels with large diameters. In contrast to stents, which serve for the treatment of vascular occlusions or constrictions, the implant can exhibit a smaller radial force, which is sufficiently substantial for the unfolded lattice structure to rest against the inside circumference of the vessel. Nevertheless, owing to its cylindrical shape it cannot be dislodged from the vein, because in the event of a theoretically insufficient unfolding, the implant is wedged at an acute angle of approximately 180 deg. at the transition from the vein to the artery owing to the change in direction of the pulmonary artery and the pulmonary vein.

A preferred embodiment of the invention provides that the constriction is formed by a necking of the tubular wall. Similarly, it is preferably provided that the wall is formed by radially expandable members. Hence, it can be provided that, in contrast, the necking is formed by a non-radially expandable member.

It is especially preferred that the members form a lattice-shaped and/or spiral-shaped structure. Owing to this structure the tubular, stent-like implant can grow into the wall of the blood vessel, so that the risk that the implant will detach itself again at a later date can be significantly reduced.

Another preferred embodiment of the invention provides that the implant exhibits anchoring mechanisms, with which the implant can be placed securely and permanently in the blood vessel. Thus, it can be guaranteed that the implant, which was once placed in situ, cannot detach itself again. Such an anchoring technique is described, for example, in the patent DE-A 203 14 392.

Other preferred embodiments of the invention provide that the constriction is configured at a distal end of the tubular wall. The terminal structure has the advantage that the constriction, which is responsible for plugging the vessel, can be pushed directly to the site that is impaired by a rupture or an aneurysm. The cross structure, which constricts the tubular lumen of the stent-like implant, reduces the cross-section lumen or the area by at least 80%, preferably by at least 90%. Therefore, a reduction by at least 95% is especially preferred. In particular, the cross section is reduced in such a manner that the blood throughflow can be totally obstructed. In one inventive embodiment the amount of throughflow can be controlled while setting the stent or at a later point in time.

It is also preferably provided that at least one wire that unfolds like a spiral or a coil is deposited in the lumen of the tubular implant. Very soon the blood platelets (thrombocytes) attach themselves to this wire (which is also called a coil), thus forming a clot by means of which the vessel is ultimately sealed, so that blood ceases to issue from the rupture or the aneurysm. The coil(s) is/are disposed in the lumen of the inventive implant; or after the inventive implant is laid or placed, the coil(s) is/are deposited in the lumen and is/are impeded from dislodging and floating away owing to the downstream constriction, which is configured at a right angle to the longitudinal direction of the tubular implant. Therefore, it is ruled out that serious subsequent complications, such as the obstruction of vital blood vessels, can be caused.

However, in this context the terminal configuration of the constriction may exhibit drawbacks. For example, it cannot be ruled out that the wire ends of the coils, which are deposited in the lumen of the tubular implant, will not pass through the constriction and/or project beyond the end of the tubular implant, thus injuring, for example, the vascular wall. If the tubular implant is arranged, for example, in the vicinity of a bifurcation, the wire ends projecting beyond the distal end of the bifurcation can protrude into the blood stream at the bifurcation and cause undesired flow turbulence.

Therefore, another preferred embodiment of the present invention provides that the constriction is configured in the region between the ends of the tubular implant, thus creating a structure that has the shape of an hourglass. Thus, it can be prevented that the wire ends protruding through the constriction can injure the vascular wall or can cause undesired flow turbulence.

This embodiment is especially preferred if the implant exhibits between its distal ends a double conical-shaped constriction, which composes a constriction or a necking, which is arranged substantially at a right angle to the longitudinal axis of the tubular, stent-like implant. The coils, which are deposited upstream as seen from the constriction, are pushed by the blood flow against the inside of the cone facing said coils. Thus, the individual wire ends are effectively prevented from passing through the constriction.

This embodiment is especially advantageous in combination with another preferred embodiment, which provides that a wire element and/or a coil in the form of a spiral-shaped double cone is deposited in the lumen or rather the hollow space of the tubular implant.

Such wire elements are disclosed, for example, in the DE-A 197 04 269. They exhibit the advantage that they can be securely deposited and locked in a tubular implant having a double conical-shaped constriction.

Moreover, these wire elements offer the advantage that they can be put together before being placed in the implant and in this state can be pushed through narrow openings, as can be arranged, for example, in a constriction according to one of the above claims. Thus, the wire element can be pushed partially through the opening and, in so doing, unfolds during the subsequent expansion on both sides of the constriction, which is at a right angle to the longitudinal direction or rather the axis of the stent-like implant. Thus, this constriction can be occluded at least almost totally.

In a preferred embodiment the wall elements of the inventive implant and/or the wire elements deposited in the lumen of the implant are made of a shape memory material. Such shape memory materials comprise preferably nickel-titanium alloys or copper-zinc-aluminum alloys.

Therefore, the unfolded shape, which corresponds to the diameter of the blood vessel to be treated, is imprinted on the blank for the inventive implant in a manner that is well-known for shape memory materials. Then the implant is compressed into a state with a small diameter and is inserted into the blood vessel by means of a catheter. In the blood vessel the implant is returned in situ to the imprinted shape by heating to the so-called conversion temperature. If desired, this unfolding process can be promoted with the use of a balloon catheter. The same procedure can also be applied to the wire elements deposited in the lumen of the implant.

Another embodiment of the invention provides that the tubular implant and/or the wire elements deposited in the lumen of the implant are made of stainless steel, platinum or plastic. Since these materials do not exhibit any shape memory properties, a tubular implant, which is made of such materials, has to be unfolded in situ by means of a balloon catheter.

In addition, it is particularly preferably provided that the surface of the inventive tubular implant and/or the wire elements is machined, in particular treated, smoothed, and/or polished. Hence, the result is a smooth surface, which satisfies in particular the requirements of the biocompatibility of the implant. The stent may be uncoated or coated by means of conventional materials in order to achieve particular biological effects.

Another especially preferred embodiment of the present invention provides that the tubular implant is made of a rigid as well as a flexible material. This rigid material may be, for example, tungsten, whereas the flexible material may be, for example, a spring steel.

In other preferred embodiments the anchoring mechanisms and/or cross section-constricting implant are made of a rigid material, whereas the remaining regions of the tubular implant are made of a flexible material.

The use of a rigid material for the locking mechanisms guarantees a better locking of the tubular implant in the vessel. The use of a flexible material for the remaining regions of the tubular implant results in the implant being easier to insert into the blood vessel and resting better against the vascular wall.

Furthermore, a method for producing an inventive implant is provided. This method is characterized in that the implant has a circumferential wall element, which is not elastically expandable in the radial direction and which in the expanded state forms a constriction of the hollow space, said constriction being configured in essence at a right angle to the longitudinal direction of the tubular implant.

Similarly, a kit of parts is provided. Said kit comprises an inventive implant and a wire element that deforms in the shape of a spiral or like a coil. Said wire element is intended for depositing in the implant and/or a balloon catheter for placing said implant, which does not expand or expands only to a diminished extent at least at one site.

Moreover, the invention provides the use of a balloon catheter for placing an inventive implant. Such balloon catheters are known, for example, for placing stents, which are used for dilation of stenoses. In this case the stent is mounted on the balloon catheter, inserted into the blood vessel to be treated, and dilated in the region which is impaired by the stenosis, in order to unfold and place in situ the stent. The present invention provides, first of all, the use of a balloon catheter for placing an inventive implant for occlusion of a blood vessel.

This feature is especially advantageous because there is a plethora of information on the correct use of balloon catheters in endovascular therapy. Thus, there is a wealth of experience, which can be referenced in order to significantly expedite the process of successfully establishing aneurysm therapy by means of the inventive implants.

The inventive implant is mounted preferably on a balloon catheter, which is inserted into the blood vessel to be treated and then is dilated in the region in which the occlusion is supposed to take place in order to unfold and, thus, to place in situ the inventive, stent-like tubular implant. Therefore, the balloon catheter is constructed preferably in such a manner that it does not expand or expands only to a diminished extent at least at one site. Thus, it is guaranteed that the tubular implant will not unfold or will unfold only to a diminished extent at this site, thus creating a constriction in the hollow space and/or the tube. Therefore, the inventive constriction, which is arranged in essence at a right angle to the longitudinal axis of the tubular, stent-like implant, is formed at this site. In particular, the aforementioned double conical-shaped embodiment of the constriction of the implant can be formed in this way.

This type of implant is suitable especially for placing tubular implants which are not made of a shape memory material.

In this context the present invention also provides a balloon catheter for placing an inventive implant. This implant is characterized in that it does not expand or expands only to a diminished extent at least at one site, thus making it possible to produce an inventive implant.

Therefore, the balloon catheter can be prepared, for example, through the previous placement of a wire loop in such a manner that it does not expand or expands only to a diminished extent at least at one site. The exact positioning of the wire loop determines the later arrangement of the resulting constriction or rather the constriction of the tubular implant. However, it is also possible to achieve by any other suitable method that the catheter does not expand at least at one site.

Another inventive method for producing an implant according to one of the preceding claims provides that the at least one spiral-like or coil-like unfolding wire or rather the wire element is deposited in the shape of a double cone in the lumen of the implant prior to the placement of the implant in the blood vessel to be treated.

Another method for depositing the wire coil or the coils provides that the at least one spiral-like or coil-like unfolding wire or rather the wire element is deposited in the shape of a double cone in the lumen of the tubular implant only after placement of the implant in the blood vessel to be treated.

In another inventive embodiment the wire elements and/or coils are configured in such a manner that they can be deposited through the lattice-shaped or spiral-shaped wall of the tubular implant into the dead space, formed between the vascular wall and the constriction or rather the lumen. Thus, it is not only impossible for the coils to dislodge, but it is also possible to control the degree of constriction in that the amount and size of the deposited coils push the wall of the implant into the vessel interior and, thus, arbitrarily constrict or also totally plug the lumen. The depositing can be done by means of the conventional catheters known to the person skilled in this art.

In another preferred embodiment of the invention, the basic structure of the tubular implant is formed in essence by a stent. Such stents are known, for example, from the DE-A 102 43 136 of the applicant of the present invention. They are inserted in tubular hollow organs in order to expand or hold said organs open and to prevent or treat, in this way, stenoses, vascular occlusions and infarctions. However, in contrast to the prior art stents, an inventive stent-like implant also exhibits a structure which is arranged in essence at a right angle to the longitudinal direction of the implant and which exhibits a constriction or necking. Furthermore, the structure can be attached to both the end which lies in the vicinity of the malformation, and to other sections of the implant. If the implant is released from the catheter, it does not obstruct just by itself the flow. The blood still continues to flow almost unobstructed through both the shunt and also through the healthy lateral branches, which are located in front of the shunt. At this stage the spirals and/or the coils can be inserted into the implant. Since the lattice-shaped structure of the implant prevents the spirals from dislodging, they may be arbitrarily undersized. This feature offers the advantage that not only a dislodgment may be prevented, but also the healthy lateral branches may be protected. If one of the struts of the implant comes to rest in front of the opening of a lateral branch, this must not be regarded as especially disadvantageous, since experience has shown that over-stenting the vessel exit sites very rarely leads to their occlusion.

A stent-like implant according to the preferred embodiment of the present invention is—just like the conventional stent—inexpensive to produce and can be just as securely placed and locked in the blood vessel as said conventional stent. Hence, the new field of application can profit from the experience that has been gathered in medicine with great success in the past from the implanting of conventional stents.

The described inventive implant, the method for producing and placing the implant as well as the method for depositing the coils are also suitable for safely disenabling an organ, preferably the spleen, by exsanguination. With some diseases it is advantageous to reduce the size of the spleen, but to retain its function. Thus, for example, the spleen may enlarge pathologically in the case of cirrhosis of the liver. Consequently, there is an increase in the blood flow in the region of the portal vein. Since, however, the blood of the portal vein cannot be discharged regularly over the liver due to the cirrhotic reconstruction of the hepatic tissue, the results are shunt circulations, for example, over the veins of the esophagus, which may lead to life-threatening hemorrhaging. In addition, an enlargement of the spleen may exert an impeding effect on the bone marrow.

In certain diseases, such as chronic lymphatic leukemia, the spleen may become extremely large and cause problems merely owing to its size. In addition, there are diseases, like Pfeiffer's glandular fever, where the spleen causes a too rapid and excessive breakdown of the red blood cells. In the case of these diseases surgical extirpation of the spleen is generally associated with a risk-entailing intervention. Attempts have been made to have a positive effect on the blood flow and/or the size of the organ by targeted embolization of the spleen. However, this, too, is subject to complications, since the embolization material, which is washed into the splenic tissue, leads to an infraction, so that such a therapy has not been able to gain acceptance.

However, an acute total occlusion of the splenic artery with coils, for example, on a level with the hilus of the spleen—that is, directly in front of the beginning of the bifurcations of the splenic artery into the segmental arteries—may also lead to serious complications in this case due to a sudden exsanguination of the whole organ. These complications can be avoided, if prior to the actual total vascular occlusion, the splenic artery could be constricted over a certain period of time with the reduced supply of blood owing to this constriction. The inventive implant can be inserted, for example, into the splenic artery with a catheter. Then, the coils are placed until the blood flow is significantly reduced, but not totally stopped. After a waiting period of a few days up to weeks, the artificially produced stenosis can be either intensified to an even higher degree or the vessel can be totally occluded in a second surgical intervention. Then complications in terms of an infarction can no longer be expected. Consequently the size of the organ will decrease while simultaneously retaining its function.

The use of an inventive implant may also serve to protect a transplanted liver. A transplanted liver may be damaged by too high a supply of portal artery blood. Therefore, in certain cases it is desirable to reduce the inflow of portal artery blood in order to prevent the freshly transplanted organ from failing. However, to date the possibility of throttling the inflow in a low-stress manner did not exist. Since the blood flowing through the spleen flows over the splenic vein into the portal artery, in these cases it would be advantageous to throttle the arterial inflow to the spleen and, thus, to reduce the blood supply of the portal artery. Such a throttling can be achieved with the inventive implant.

Various embodiments of the invention are explained in detail below with reference to the figures.

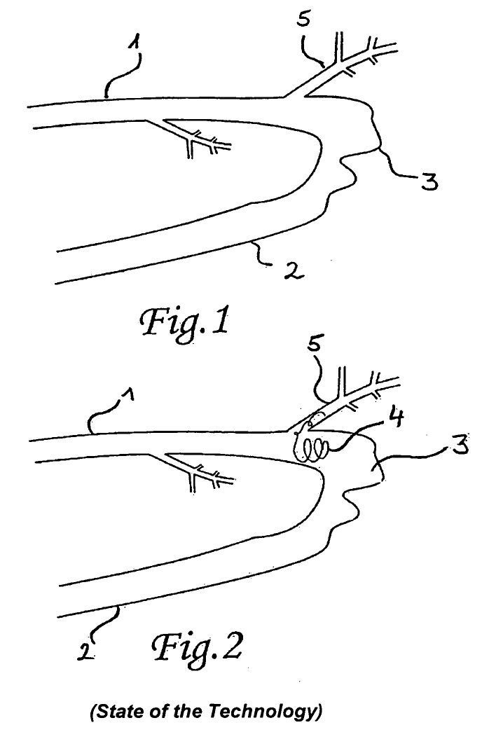

FIG. 1 depicts an arteriovenous malformation with shunts between the pulmonary artery and the pulmonary vein.

FIG. 2 depicts a method from the state of the art for depositing a coil into such a malformation.

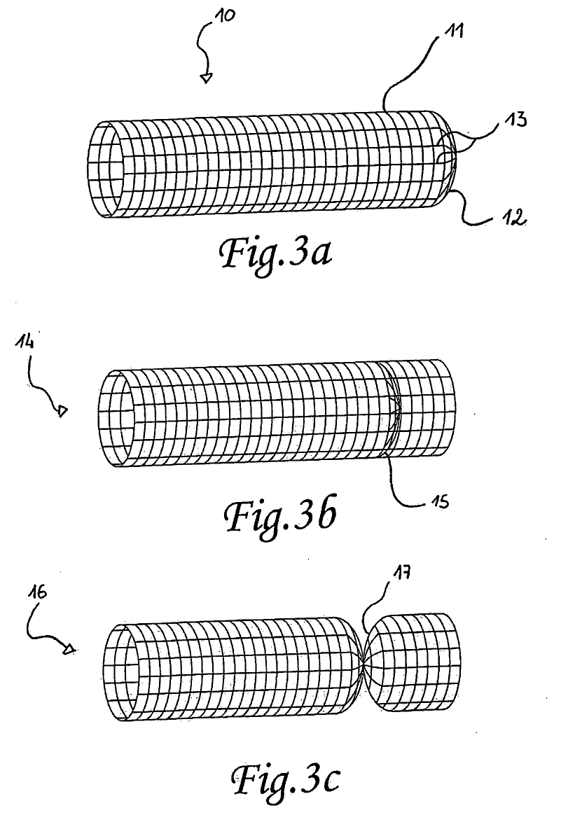

FIG. 3 is a three dimensional view of three inventive endovascular implants.

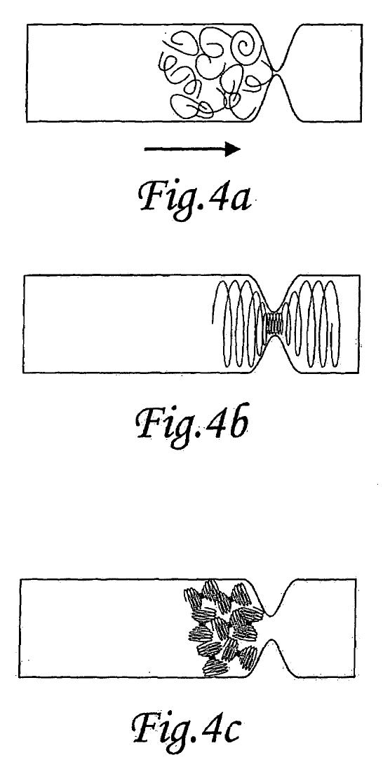

FIG. 4 is a cross sectional view of three inventive endovascular implants.

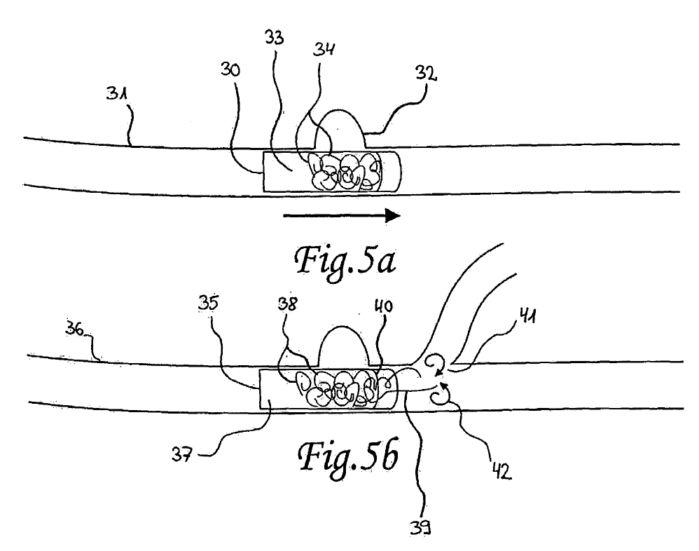

FIG. 5 depicts two inventive endovascular implants, disposed in a blood vessel, exhibiting an aneurysm.

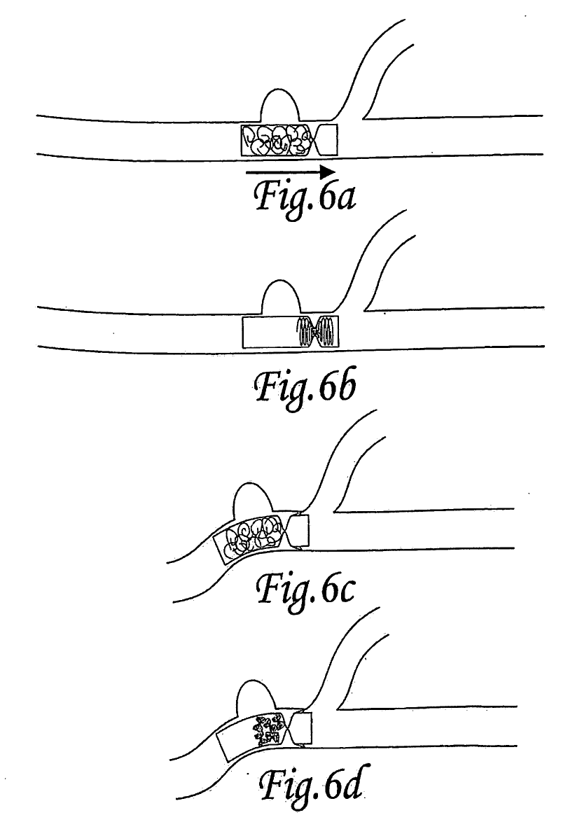

FIG. 6 depicts four inventive endovascular implants, disposed in a blood vessel, exhibiting an aneurysm.

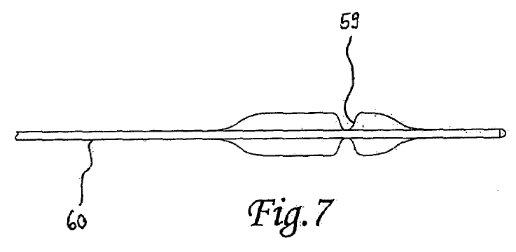

FIG. 7 depicts a balloon catheter for placing and unfolding an inventive endovascular implant; and

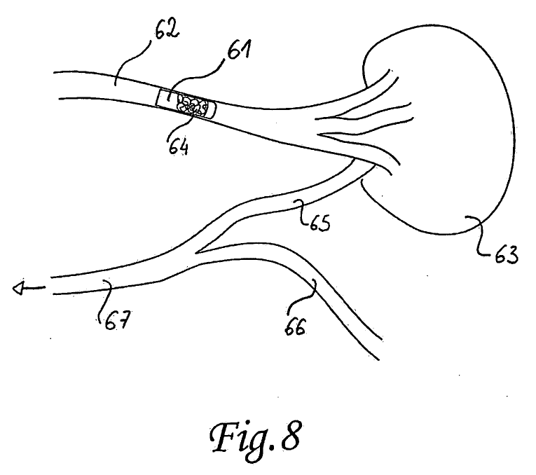

FIG. 8 depicts the use of an inventive implant in the splenic artery.

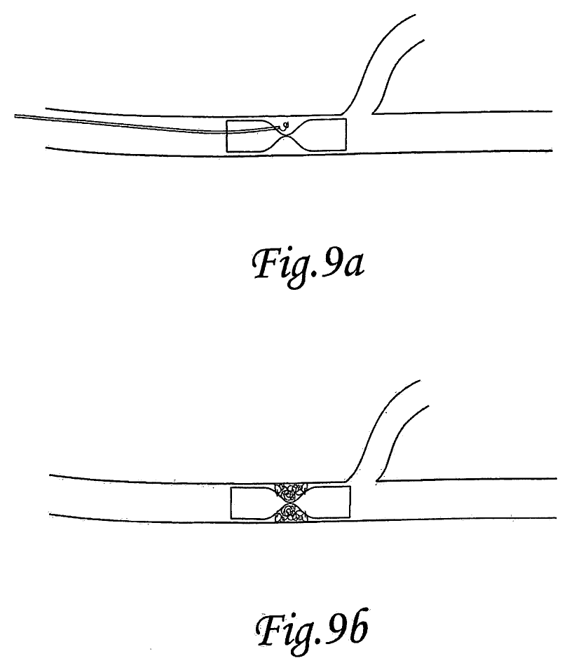

FIG. 9 depicts the inventive depositing of coils and/or wire elements in a dead space (a) formed between the vascular wall and the wall of the implant, as well as the occlusion (b) that is produced with said coils and/or wire elements.

FIG. 1 depicts an arteriovenous malformation with shunts between the pulmonary artery 1 and the pulmonary vein 2. A malformation 3 itself is characterized by an often glomerular, sometimes aneurysm-like expansion of the vessel, which, after a 180 deg. change in direction, continues then as the vein 2 in the sense of a shunt and goes directly to the left atrium of the heart. Since it is a shunt, a high blood flow usually prevails in the afferent branch.

FIG. 2 depicts a method from the state of the art for depositing a coil into such a malformation. In this case an attempt is made to anchor a spiral 4 with its lead section in a healthy efferent lateral branch 5, so that the major portion of the spiral can lie in the pathologically expanded vessel segment 3 without being dislodged, even if the spiral is relatively undersized. Then the subsequent spirals are deposited directly into the expanded vessel. Thus, the first spiral 4 serves to block the flow and then to bring the following spirals into a stable position.

FIG. 3a is a three dimensional view of an inventive endovascular implant 10, which comprises a tubular implant 11, which exhibits a structure 12, which is arranged in essence at a right angle to the longitudinal direction of the implant. The implant exhibits a lattice-shaped wall structure, which can be configured, for example, in the form of a wire mesh.

The hollow space constriction 12 is arranged at the one end of the implant 10 and is configured as a fine netting 13, to which upon arranging the implant in a blood vessel the blood platelets (thrombocytes) attach very soon and, thus, form a clot, by means of which the vessel is ultimately occluded. However, the constriction itself can also be configured in such a manner that it alone securely plugs the blood vessel.

FIG. 3b is a three dimensional view of an inventive endovascular implant 14, comprising a structure and/or constriction 15, which is arranged in essence at a right angle to the longitudinal direction of the implant and which is arranged in the region between the ends of the implant 14. The constriction 15 is configured as a fine netting, on which a clot forms in a manner that is well-known. However, the constriction itself can also be configured in such a manner that it alone securely plugs the blood vessel.

FIG. 3c is a three dimensional view of an inventive endovascular implant 16, comprising a structure and/or constriction 17, which is arranged in essence at a right angle to the longitudinal direction of the implant and which owing to the formation of a necking forms a double conical-shaped constriction of the wall of the implant 20.

FIG. 4a is a cross sectional view of the inventive endovascular implant 20, which is shown in FIG. 3c and which comprises a structure 21, which is arranged at a right angle to the longitudinal direction of the implant and which is formed by the formation of a double conical-shaped constriction of the wall of the implant 20. The arrow shows the direction of the blood flow in arranging the implant 20 in a blood vessel.

A plurality of spiral-like or coil-like unfolding wires are deposited in the lumen 22 of the implant 20. When the implant 20 is disposed in a blood vessel, blood platelets attach themselves very soon to these wires, which form a large surface and are also referred to as coils 23, and, thus form a clot, by means of which the vessel is ultimately plugged. The coils 23 are disposed in the lumen of the implant 20 and are pushed by the blood flow against the inside of the downstream cone of the constriction 21 that faces the coils. Therefore, the coils are prevented from dislodging and floating away. Thus, it is ruled out that they can cause serious subsequent complications, such as an obstruction of a coronary artery. In addition, the conical shape effectively prevents the individual wire ends of the coils 23 from passing through the constriction 21.

FIG. 4b is a cross sectional view of the inventive endovascular implant 24, which is depicted in FIG. 3c and which comprises a structure 25, which is arranged at a right angle to the longitudinal direction of the implant. The arrow shows the direction of the blood flow in arranging the implant 24 in a blood vessel.

A wire element 27 in the shape of a double cone is deposited in the lumen 26 of the implant 24. Such wire elements exhibit the advantage that they can be securely deposited and locked in an implant 24 exhibiting a double conical-shaped constriction 25. Moreover, these wire elements offer the advantage that they can be put together before being placed in the tubular implant and in this state can be pushed through narrow openings, as can be arranged, for example, in the constriction 25. Thus, the wire element 27 can be pushed partially through the opening and, in so doing, unfolds during the subsequent expansion on both sides of the constriction 25.

The double conical-shaped wire element 27 enlarges the surface and increases the turbulence. Thus, the blood platelets coagulate faster and, thus, form a clot, by means of which the vessel is ultimately plugged.

FIG. 5a depicts an inventive endovascular implant 30, disposed in a blood vessel 31, exhibiting an aneurysm 32. The arrow shows the direction of the blood flow. The implant 30 exhibits coils 34, which are deposited in the lumen of the implant and to which the blood platelets attach themselves and thus, form a clot, by means of which the vessel 31 is ultimately plugged. In this way a rupture of the aneurysm is prevented; and/or following a rupture, the blood is prevented from issuing from the blood vessel 31 and possibly causing a life-threatening situation, such as subarachnoidal bleeding.

FIG. 5b depicts an inventive endovascular implant 35, disposed in a blood vessel 36. The implant 35 exhibits coils 38, which are deposited in the lumen 37 of the implant and whose ends 39 pass to some extent through the constriction 40 and project into the blood stream at a bifurcation 41, where they can cause an undesired flow turbulence 42.

FIG. 6a depicts an inventive endovascular implant 43, disposed in a blood vessel 44. The implant 43 exhibits a constriction 45, which is formed by the formation of a double conical-shaped constriction of the wall of the implant 43. A plurality of spiral-like or coil-like unfolding coils 47 are disposed in the lumen 46 of the implant. A clot forms in the described manner on said coils and, thus, ultimately plugs the vessel 44. The coils 47 are disposed into the lumen of the implant 43 and are pushed by the blood flow against the inside of the downstream cone of the constriction 45 that faces the coils. Therefore, the coils are prevented from dislodging and floating away. In addition, the conical shape effectively prevents the individual wire ends of the coils 47 from passing through the constriction 45. Hence, it is ruled out that the wire ends can project into the blood stream at a bifurcation 48, where they can cause an undesired flow turbulence.

FIG. 6b depicts an inventive endovascular implant 49 disposed in a blood vessel 50. The implant 49 exhibits a constriction 51, which is formed by the formation of a double conical-shaped constriction of the wall of the implant 49. A wire element 53 in the shape of a double cone is disposed in the lumen 52 of the implant 49. A clot forms in the described manner on said wire element, and thus ultimately plugs the vessel 50.

The wire element can be securely deposited and locked in the implant 49, comprising a double conical-shaped constriction 51, and can be put together before being placed in the tubular lumen and in this state can be pushed through narrow openings, as formed, for example, in the constriction 51.

FIG. 6c depicts an inventive endovascular implant 54, disposed in a blood vessel 55. The implant 54 exhibits a constriction 56, which is formed by the formation of a double conical-shaped constriction of the wall of the implant 54. A plurality of spiral-like or coil-like unfolding coils are disposed in the lumen 57 of the implant. A clot forms in the described manner on said coils, thus ultimately plugging the vessel 55.

The implant 54 exhibits locking mechanisms 58, which are made—just like the constriction 56—of a rigid material. In contrast, the remaining regions of the implant 54 are made of a flexible material. The use of a rigid material guarantees a better locking of the implant 54 in the blood vessel 55. The use of a flexible material for the remaining regions of the implant 54 results in the implant 54 being easier to insert into the blood vessel 55 and resting better against the vascular wall.

FIG. 7 depicts a balloon catheter 60, which is shown in the expanded state and is intended for placing and unfolding an inventive endovascular implant.

The catheter resembles conventional catheters for the placement and unfolding of stents, as used for dilation of vascular stenoses. However, the balloon catheter is constructed in such a manner that it does not expand at a site 59. Thus, it is guaranteed that an implant (not illustrated), which is mounted on said catheter, will not unfold at this site. Thus, the implant forms a constriction, as already described above.

For this purpose the balloon catheter exhibits a wire loop (not illustrated), which is slid over said balloon catheter and which prevents in situ the expansion. The exact positioning of the wire loop determines the subsequent arrangement of the resulting constriction of the implant.

FIG. 8 depicts the use of an inventive implant 61 in the splenic artery 62, in order to reduce, for example, the size of the spleen 63, but to retain its function (which is advantageous for some diseases (see above)), or to reduce the inflow of portal artery blood into the liver. The implant is inserted into the splenic artery 62 by means of a catheter; and then the coils 64 are placed until the blood flow is significantly reduced, but not totally stopped. After a waiting period of a few days up to weeks, the artificially produced stenosis can be either intensified to an even higher degree or the vessel can be totally occluded in a second surgical intervention. Then complications in terms of an infarction can no longer be expected. In this case the size of the spleen 63 will decrease while simultaneously retaining its function.

Similarly the blood flow in the splenic vein 65, which empties into the mesenteric vein 66, will decrease; and thus the blood flow in the portal artery 67, which passes into the liver, will also decrease. In this way the inflow of portal artery blood into the liver can be reduced, a feature that is advantageous, for example, in post-surgical treatment of a liver transplant.

FIG. 9 depicts an inventive procedure using the inventive implant. In this case in order to achieve a partial or total occlusion of a vessel, an inventive stent is deposited in the vessel at an arbitrary desired location by means of a catheter. As the inventive stent expands, an hourglass-shaped constriction forms in the vascular lumen. Thus, a dead space is created between the vascular wall and the hourglass-shaped constriction. However, this dead space is not totally sealed by the open lattice structure, which can also exhibit, if desired, a spiral-shaped structure. Through these lattice openings, [coils] are brought into the dead space by means of a thin catheter, which is preferably a coaxial catheter. Such coaxial catheters are used in radiology and are known to the person skilled in this art. They usually exhibit an outside diameter of approximately 1 mm. According to the invention, the dead space is now filled with small spirals by means of this coaxial catheter. Owing to this inventive procedure or inventive use of the stent, it is also possible to use non-jacketed (non-covered) stents. In particular, a coating causes the stent to be relatively rigid or stabler. In addition, the manufacture of coated stents is considerably more expensive and, thus, raises the production costs. Finally, the inventive procedure also makes possible a variable control of the throughflow of the blood vessel. Thus, with fluoroscopic screening, for example by means of x-rays, the reduction of the throughflow can be observed directly and—by depositing additional spirals—can be limited to the desired degree as far as and including total occlusion.

1. Endovascular, stent-like expanding implant (10), in particular, for occlusion of a blood vessel (31), said implant comprising a circumferential tubular wall, which extends about a longitudinal axis and is made of a radially expandable, flexible lattice structure, wherein the wall encloses a cylindrical hollow space with a cross section, characterized in that the cylindrical hollow space, which is defined by the wall, exhibits a constriction at which the blood vessel can be plugged, at least at one location along the axis of said hollow space.

2. Implant as claimed in claim 1, characterized in that the constriction is formed by a necking of the tubular wall.

3. Implant as claimed in any one of the preceding claims, characterized in that the wall is formed by radially expandable members.

4. Implant as claimed in any one of the preceding claims, characterized in that the implant exhibits anchoring mechanisms, with which the implant can be placed securely and permanently in the blood vessel.

5. Implant as claimed in any one of the preceding claims, characterized in that at least one wire (23) that unfolds like a spiral or a coil is deposited in the lumen of the tubular implant.

6. Implant as claimed in any one of the preceding claims, characterized in that the wall elements of the implant and/or the wire elements (23, 27) deposited in the lumen of the implant are made of a shape memory material.

7. Implant as claimed in any one of the claims 1 to 5, characterized in that the tubular implant and/or the wire elements deposited in the lumen of the implant are made of a material that is selected from the group, comprising stainless steel, platinum and plastic.

8. Method for producing an implant as claimed in any one of the claims 1 to 7, characterized in that the implant has a circumferential wall element, which is not elastically expandable in the radial direction and which in the expanded state forms a constriction of the hollow space, said constriction being configured in essence at a right angle to the longitudinal direction of the tubular implant.

9. Kit of parts comprising an implant, as claimed in any one of the claims 1 to 7, as well as a wire element that deforms in the shape of a spiral or like a coil and which is intended for depositing in the implant and/or a balloon catheter for placing an implant, which does not expand or expands only to a diminished extent at least at one site.

10. Application of an implant as claimed in any one of the claims 1 to 7, for reducing and/or blocking the flow of blood through an organ.
